(19) United States
(12) Patent Application Publication
(10) Pub. No.: US 2021/0193325 A1
(43) Pub. Date: Jun. 24, 2021

(54) SYSTEM AND METHOD FOR PROVIDING A DRUG THERAPY COORDINATION RISK SCORE AND IMPROVEMENT MODEL-OF-CARE

(71) Applicant: CareOregon, Inc., Portland, OR (US)

(72) Inventor: James William Slater, Beaverton, OR (US)

(73) Assignee: CareOregon, Inc., Portland, OR (US)

(21) Appl. No.: 17/191,494

(22) Filed: Mar. 3, 2021

Related U.S. Application Data
(63) Continuation-in-part of application No. 15/788,785, filed on Oct. 19, 2017, now abandoned.
(60) Provisional application No. 62/410,161, filed on Oct. 19, 2016.

(51) Int. Cl. G16H 50/30; G16H 20/10; G16H 10/60

(52) U.S. Cl. CPC G16H 50/30; G16H 10/60; G16H 20/10

(57) ABSTRACT

The present invention generally relates to pharmacy claim data processing, and in particular it relates to coordination scoring, patient profiling, patient and prescriber behavior analysis and modeling. More specifically, it relates to coordination of medication use risk modeling using the inputs of pharmacy claims data, prescriber data, and, optionally, medical claims data.

### TECHNICAL FIELD

This description relates generally to the coordination of the dispensation of pharmaceutical products (such as prescription drugs), and more specifically to improving coordination in prescribing and administering drugs to reduce risk of adverse effects in drug therapy.

### BACKGROUND

Prescription medications (as well as non-prescription medicines) can interact-often with adverse consequences to a patient. Alternatively some medications may interfere with the effectiveness of other medications being taken. The administration of medications may be somewhat disorganized due to the presence of multiple health care providers and the like. A single health care provider administering multiple medications to a patient to treat one or more ailments is challenged to deal with undesired side effects and drug interactions. The situation is further aggravated when multiple health care providers may be involved, who further may not be aware of other health care providers medications the patient may be taking. Often a pharmacist or other health care provider who knows of some or all of a patient's multiple medications may notice medications with undesirable interactions through review, or by chance. Aside from adverse effects, different drugs may interfere with the successful function of a particular drug, reducing its effectiveness in treating a given health problem.

Such fragmented medication management can create patient confusion and anxiety, resulting in what may be termed “medication trauma”. Medication trauma is typically the result of medication complexity, and a lack of coordination that can overwhelm the patient, caregivers, and other provider resources. Such medication trauma can result in creating fear, confusion, and error which further leads to poor adherence, compliance and outcomes with respect to the patient's medications being taken. The current state of medication prescribing practice typically allows patients to receive multiple medications from multiple prescribers with somewhat haphazard or non-existent formal oversight. Multiple, and often interfering medications may require multiple medication changes, and frequent pharmacy visits to stay compliant and adherent with medications. In addition the patient may not know why they are taking a particular medication, may not know if it is working, and may feel rushed or uncertain as to how to communicate with their health care provider. The patient who might be assumed to be ultimately responsible for their own health might be impaired due to age, infirmity, the medication they are taken, and their own ability to comprehend what they need to do to maintain their health. This may lead to increased risk, by not adhering to diet and exercise requirements, leading to worsening of their disease. Worsening of the disease can lead to further medication increase which can exacerbate the medication trauma caused to a patient as well. Such a cycle can be a vicious circle that leads to increased ER and hospitalization, to the detriment of the patient and increased health care costs.

Prescribers and pharmacists have historically lacked a method to identify and quantify medication coordination risk and prioritize the use of healthcare resources in a constructive, progressive way to improve patient experience and outcomes with medication coordination. A system and method that assesses risk, and accordingly operates to reduce risk to the patient, lessen health care costs, and possibly improve treatment outcomes would be desirable.

### SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

A risk score may be obtained by examining pharmacy data and using it in a predictive model. Once a score is obtained patients having a risk score above a threshold value may be given increased scrutiny in their care. The risk score may be made known to the patient and others by printing it on various items, such as prescription medicine labels, medical records and the like. The labeling may be produced by devices such as dedicated thermal printers that may be loaded with blank labels, blank tamperproof labels, or the like if desired. Proliferating the score with labeling raises awareness with health care providers regarding the prescription risk for a given patient suggesting greater scrutiny may be needed regarding their medications, at least more so than one with a lower risk score. Further, to reduce a score for a patient the inputs may be examined for improvement, and the risk score recalculated in an effort to reduce the risk in a quantifiable manner.

Unlike other methods of identifying patient risk that typically require various sources of data to identify risk, the systems and methods described herein uniquely only need to take into consideration easily obtained pharmacy data in order to determine a risk score and share it with the patient. Patients at risk for medication trauma can be identified through such pharmacy claim patterns, or other suitable pharmacy data. Additional data sources may be utilized to provide further accuracy in scoring. Untreated medication trauma may lead to higher Emergency Department (“ED”) and hospital utilization as a result of medication interactions. Once scored treatment may be rescored and reevaluated to determine if the risk has been reduced, or if further refinement in treatment is needed.

Many of the attendant features will be more readily appreciated as the same becomes better understood by reference to the following detailed description considered in connection with the accompanying drawings.

### DESCRIPTION OF THE DRAWINGS

The present description will be better understood from the following detailed description read in light of the accompanying drawings, wherein:

FIG. 1 shows a process for managing health care utilizing a drug therapy coordination risk score.

FIG. 2 is an exemplary user interface diagram that may be utilized in a health care management system utilizing a drug therapy coordination risk score.

FIG. 3 is a diagram illustrating an exemplary drug therapy coordination plan.

FIG. 4 is a graph plotting drug therapy coordination risk score against time for an exemplary pre-intervention patient.

FIG. 5 is a graph plotting drug therapy coordination risk score against time for an exemplary patient of FIG. 5 after intervention.

FIG. 6 illustrates an exemplary computing environment in which the system and method for providing a drug therapy coordination risk score and an improved model of care (“pharmacy risk model”) described in this application, may be implemented.

FIG. 7 is an exemplary network in which the system and method for providing a drug therapy coordination risk score and an improved model of care (“pharmacy risk model”) may be implemented.

Like reference numerals are used to designate like parts in the accompanying drawings.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

The examples below describe a system and method for providing a drug therapy coordination risk score and an improved model of care with a “pharmacy risk model”. Although the present invention is described as relating to risk modeling of individual patients, one of skill in the pertinent arts will recognize that the various embodiments of the invention can also apply to drugstores, heath plans, pharmacy benefit management companies, health care clinics, health care organizations, and the like without departing from the spirit and scope of the present invention.

Unlike other methods of identifying patient risk that typically require various sources of data to identify risk, the systems and methods described herein uniquely only need to take into consideration pharmacy data in order to determine a risk score. The present examples allow any component of a healthcare system with access to core medication use data (pharmacy data) to create a risk model for use in targeting patients; prioritizing resources regarding coordination of medication of at risk patients, and generally increasing medication coordination among providers to improve healthcare outcomes. A method of obtaining a risk is provided, and additionally a method for improving the risk score is described.

Patients at risk for medication trauma can be identified through pharmacy claim patterns. The use of easily obtainable pharmacy claim data sets this system and method apart from other methods that may typically require inputs from other sources that may be hard to obtain and quantify. Untreated medication trauma typically leads to higher ED and hospital utilization. Accordingly the systems and methods described herein tend to evaluate the risk of medication trauma using pharmacy claim data, so that risk may be decreased in a quantifiable manner.

Based on such use patterns a method for determining a comprehensive medication coordination default risk value for a patient has been constructed. The risk value may be obtained and evaluated by various methods including those utilizing software, firmware and hardware-such as programing the risk model process into computing devices such as a hard wired logic circuit, programmable logic array or the like. The score may be communicated for instutional use typically through transfer over a computer network. Additionally, the score may be communicated by simple printing on various forms, documents and labels. In particular a patients medication labels may be marked with their risk value, so that care givers may exercise appropriate caution in administering existing medications and in prescribing additional medications.

Alternative examples of the method may include obtaining medical claim data and internal data relating to the patient and prescribers and further defining the prioritization of healthcare resources and staff to improve comprehensive drug therapy coordination default risk value for the patient based upon said internal data. However the pharmacy claim data has tended to produce a typically highly accurate score, without needing further data from alternate additional sources.

The pharmacy risk model results may be utilized to aid medication trauma risk patients by:
identifying and empaneling high risk medication trauma patients;
establishing a coordinated network of pharmacists to support these patients;
improving and innovating the role of pharmacists through a primary care pharmacist collaborative;
providing reliable medication support to patients undergoing transitions-of-care; and
advancing medication education and workflows around actionable patterns required to stabilize or prevent medication trauma.

Modeling patient drug therapy coordination risk includes, in one embodiment, obtaining pharmacy claim data, modeling and/or processing the pharmacy claim data, and creating an output for evaluation. The output may then be used to make healthcare resource allocation decisions and patient screening and management decisions to improve coordination and success with medications. In various embodiments, the present invention uses a variety of data (e.g., pharmacy claim data) in conjunction with several modeling/processing procedures to assess risk.

Creating a pharmacy risk score using only pharmacy claims to predict ED and hospitalization using pharmacy coordination data as important factor to predict risk.

Today there is not a quality measure for medication coordination. The method of determining a pharmacy risk score otherwise know as Drug Therapy Coordination Risk Score provides and objective way to measure coordination risk as a quality measure.

The measure also serves and provides unique insight into healthcare operations of the providers creating prescriptions, as it shows the clinics operations efficiency and ability to manage and coordinate patient populations being served. Accordingly this score may also serve as a proxy for clinic operational efficiency and quality-of-care.

A Pharmacy Quality Alliance or a major healthcare organization can adopt the risk model to improve medication coordination, reduce trauma and improve provider and member experience.

### DESCRIPTION

The Drug Therapy Coordination Risk (“DTCR”) score method identifies patients with the highest risk of inpatient and ED utilization. This method is unique because it solely utilizes pharmacy variables.

### Methodology

The method was developed using pharmacy claims data from a medical organization's adult members having the highest inpatient services and ED usage (“High Utilizers”). The data is based on the examination of the records of approximately 80,000 members of the organization. The DTCR scores of this High Utilizer group was analyzed, and it revealed a correlation between high DTCR scores and high inpatient services and ED usage. This observation was validated by analyzing the DTCR scores calculated for the entire adult population of health care organization's adult members. It was verified that DTCR scores increased with increased inpatient services and ED usage.

The model utilizes a Dependent Variable Utilization Index, which is a weighted composite of Inpatient stays and ED visits. The independent variables are derived from pharmacy claims data. What differentiates the model from other models that currently exist in the industry is that its drivers originate solely from pharmacy claims data. Due to the fast claims processing time, pharmacy claims data often serves as an early warning indicator of high risk individuals who could benefit from further medical care intervention.

The exemplary model was built using Ordinary Least Squares in SAS (Statistical Analysis Systems www.sas.com). During the course of model development, hundreds of variables based on pharmacy claims data were input into a statistical procedure known to those skilled in the art called PROC GLMSELECT. The procedure selected the best variables based on a criterion of R-squared optimization. R-squared is a number between 0 and 1 and represents the explanatory power of the model.

Although SAS was the tool utilized for the development of the model, the model is easily transportable to other statistical modeling tools known to those skilled in the art such as SPSS, STATA, or R, which is a free statistical package widely used by the research community.

In examples of the invention the risk modeling may be performed by hard wired circuitry, such as ASICs or the like that implement the logic function. Also, access to the risk model may be provided through identity readers custom made by methods known to those skilled in the art to provide a desired level of security and prevent unauthorized access.

### Method

An algebraic formula for risk scoring was derived and tested by using pharmacy claims data to score adult members of a health care organization. Threshold values of DTCR score were created and used to identify patients who could benefit from a pharmacy or medical intervention to achieve better health outcomes. The algebraic formula for calculating a patient's DTCR risk score is shown below in equation (1).

DTCR Score=4.5−0.04(Distinct Fill Date)+0.34(Distinct GPI14Count)−0.31(Distinct GPI2Count)+0.31(Distinct Pharmacy Count)+0.56(Distinct Prescriber Count)−0.06(Average Days Supply) (1)

Where:
“Distinct Fill Date” is defined as Date Prescription was Filled;
“Distinct GPI14Count” is defined as Generic Product Identifier from Medi-Span; a hierarchical therapeutic classification structure;
“Distinct GPI2Count” is defined as derived from GPI14. GPI14 has the most detail. GPI2, the first two characters, defines the drug group;
“Distinct Pharmacy Count” is defined as the pharmacy's count;
“Distinct Prescriber Count” is defined as the prescriber's count;
“Average Days' Supply” is defined as Days of Drug Supply (eg 30 days);
and 4.5 is an adjustment factor suggested by the model.

The above variables are those commonly obtained from pharmacy claim data, and the variable names may vary in alternative examples of pharmacy claim data. Accordingly, a higher DTCR score correlates to a higher risk and higher potential to benefit from intervention. Typically a score of typically 8 or greater may be considered to be indicative of a high medication trauma risk. Patients with certain scores may be diverted to various medication management programs to lessen their risk. For example patients with a score of 9-14.99 may be referred to an exemplary medication management program, and those with a score 15 or above to an exemplary intensive medication management program.

The following is exemplary SAS code for the pharmacy risk model:

Example: Hypothetical Patient X, has the following values from his pharmacy claims data: Distinct Fill Date=10, Distinct GPI14Count=6, Distinct GPI2Count=2, Distinct Pharmacy Count=4, Distinct Prescriber Count=3, and Average Days' Supply=30.

Applying formula (1), calculation of Patient X's DTCR score is shown below.

DTCR Score=4.5−0.04(10)+0.34(6)−0.31(2)+0.31(4)+0.56(3)−0.06(30) (2)

DTCR Score=6.64 (3)

### Model Results

The model's pharmacy-based independent variables are able to explain 0.29 percent of the variance (R-squared) in Utilization Index. Table 1 shows the model's five independent variables, along with their coefficients and t-value.

TABLE 1
Model Results:
Dependent Variable: Utilization Index (Adj. R-squared: .29)

| | | Standard | |
|---|---|---|---|
| Independent Variable | Estimate | Error | t Value |
| Intercept | 4.5 | 0.21 | 20.8 |
| Distinct Fill Dates | -0.04 | 0.003 | -12.1 |
| Distinct GPI14 Count | 0.34 | 0.02 | 15.4 |
| Distinct GPI2 Count | -0.31 | 0.03 | -8.9 |
| Distinct Pharmacy Count | 0.31 | 0.04 | 7.3 |
| Distinct Prescriber Count | 0.56 | 0.02 | 27.9 |
| Average Days Supply | -0.06 | 0.01 | -7.4 |

This model compares favorably with a baseline model based on a prior art system. The prior art system assigns patients a risk score based on complete medical data including medical and drug claims and clinical information. Notably this method requires data in addition to the simple pharmacy data only required in the current examples. Data other than pharmacy data is not required. While, the ACG risk score, recognized by the health care industry, explains 11% of the variance in Utilization Index, the DTCR pharmacy-based model described herein reflects a 29% Utilization Index.

Unlike other models, the DTCR model is outstanding in its simplicity and interpretability. Its five drivers can be easily collected from pharmacy claims data. The model can also be used to score new patients based on self-reported measures of pharmacy utilization.

The following figures describe exemplary computing systems in which the risk model may be implemented. In each of these systems specially constructed and dedicated hardware may be provided to implement and secure the risk model, typically with HIPPA level security to protect patient confidentiality.

Finally with regards to implementing a training model, it has been found that in regard to biasing the model, that less bias may be introduced by including all adult members rather than high utilizers, since they are typically a small subset of the entire population.

The present description will be better understood from the following detailed description read in light of the accompanying drawings, wherein:

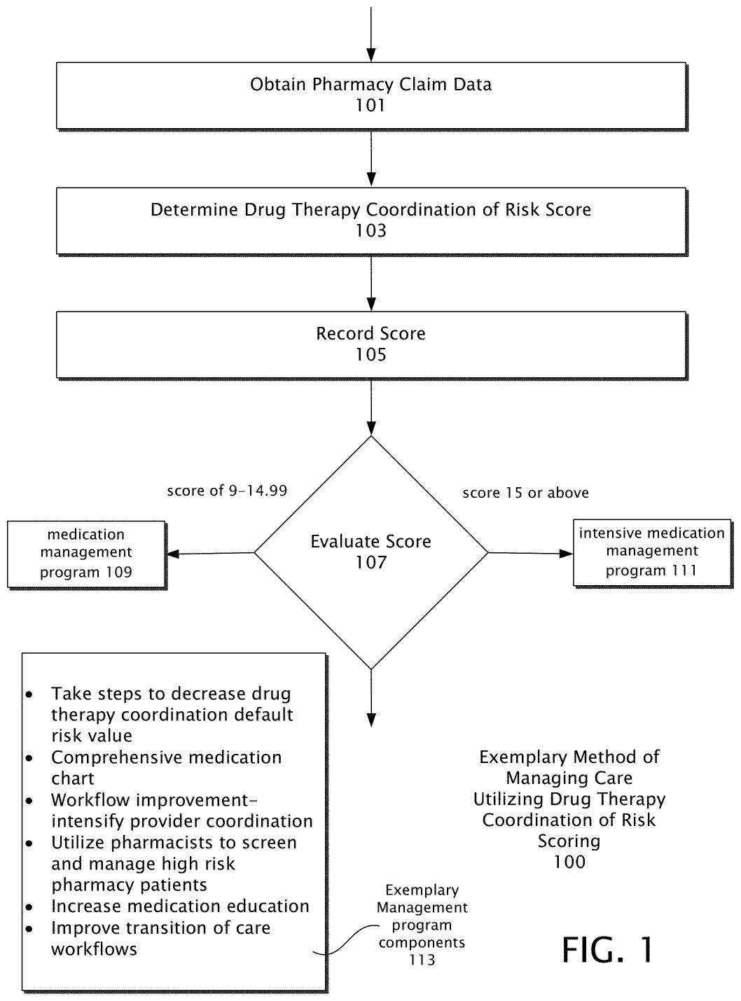

FIG. 1 shows a process for managing health care utilizing a drug therapy coordination risk score. At block 101 pharmacy data is obtained, and information needed to determine a drug therapy coordination risk score is obtained. At block 103 the drug therapy coordination risk score is determined utilizing equation (1). At block 105 the score may be recorded in a data base. At block 107 the score is evaluated to determine if intervention, and the level of intervention that might be needed. For example if the score falls within an exemplary first range of 9-14.99 referral to a first program such as the exemplary medication management program 109, may be in order. If the score falls within an exemplary second range and is of an exemplary range of 15 or above, then the patient may be referred to an intensive medication management program 111. Part of the intensive medication management program may include printing labels and reports that include the drug therapy coordination risk score. Having prescription medication labeled with the drug therapy coordination risk score helps increase awareness with the consumer and caregivers regarding the prescription drug interaction risks a patient may be facing. Also in case someone might wish to downplay risk a patient may be facing (including the patient themselves) the labels may be of the tamper resistant type that may be loaded into an exemplary thermal printer, or the like. Alternatively any number of referrals may be utilized based on score, or range of score. Some components of the various possible management programs are also listed 113 for reference.

In alternative examples it is important to know that while the variables used have not changed, the model may update the modifiers for each variable every month as 1 month falls off and a new month joins the 12 month data set. This typically allows for minor ongoing modifications. It has been found it changes very little but does allow for ongoing adjustments. In a further alternative example it has been determined that Point B analysis suggested that one variable may be removed without appreciably affecting accuracy-typically the GPI2 count, since it is typically highly correlated to the GPI4 count. In a yet further alternative example independent variables may be normalized before running a regression analysis, since regression estimates typically have an underlying assumption of linearity in independent variables. Accordingly a Gaussian normalization of independent variables may be performed.

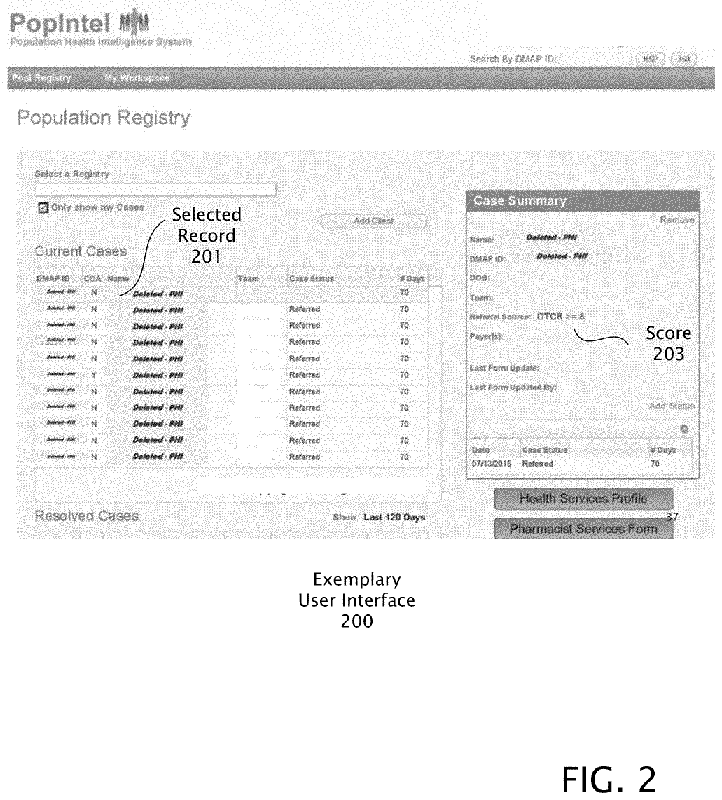

FIG. 2 is an exemplary user interface diagram 200 that may be utilized in a health care management system utilizing a drug therapy coordination risk score. Here the data base is accessed to show multiple records. In particular a selected record 201, has associated with it a score 203.

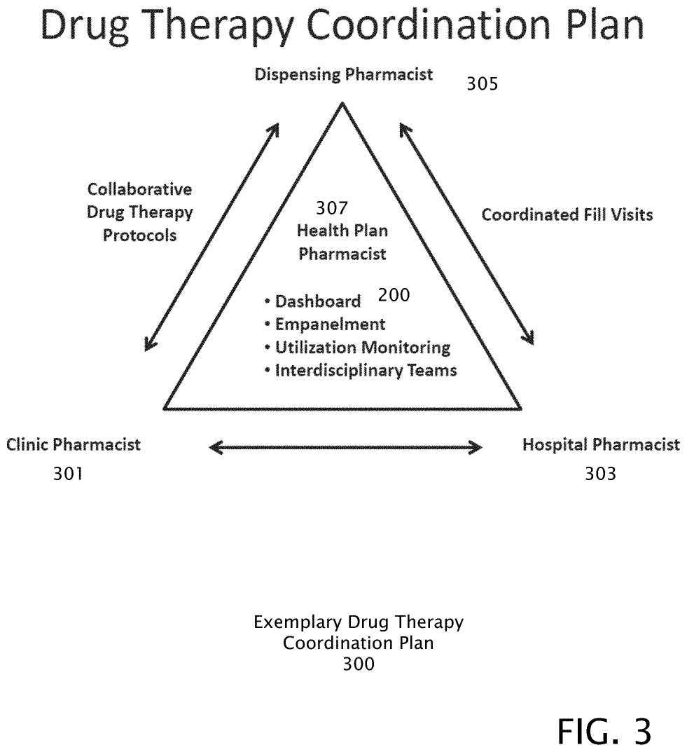

FIG. 3 is a diagram illustrating an exemplary drug therapy coordination plan 300. The dashboard 200 or other suitable interface for accessing scoring data may be used in conjunction with coordination drug therapy. In addition the data dashboard data may be provided in all or part to prescription labels, reports and the like. In other words the drug therapy coordination risk score may be accessible to healthcare providers and the patient without requiring the presence of a computer to look up the score. The score may be supplied in printed form. In particular the dashboard data 200 may be available for sharing with a health plan pharmacist 307, a dispensing pharmacist 305, a clinical pharmacist 301, a hospital pharmacist, or the like (also other branches of health care professions can access the data). Here in this exemplary model of oversight and care pharmacists have been utilized to monitor and improve scoring for patients.

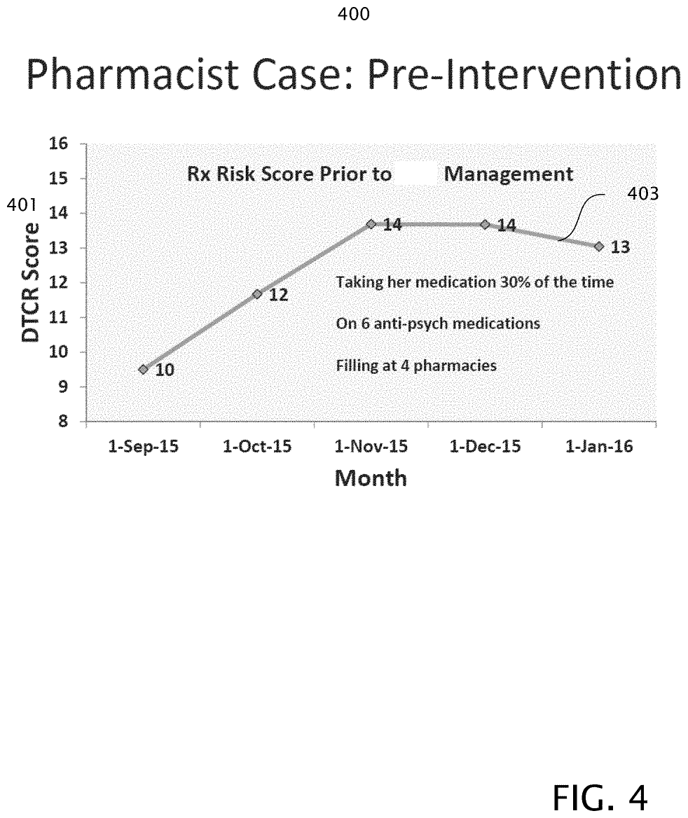

FIG. 4 is a graph plotting drug therapy coordination risk score against time for an exemplary pre-intervention patient 400. Here score 401 is plotted 403 against time for an exemplary patient.

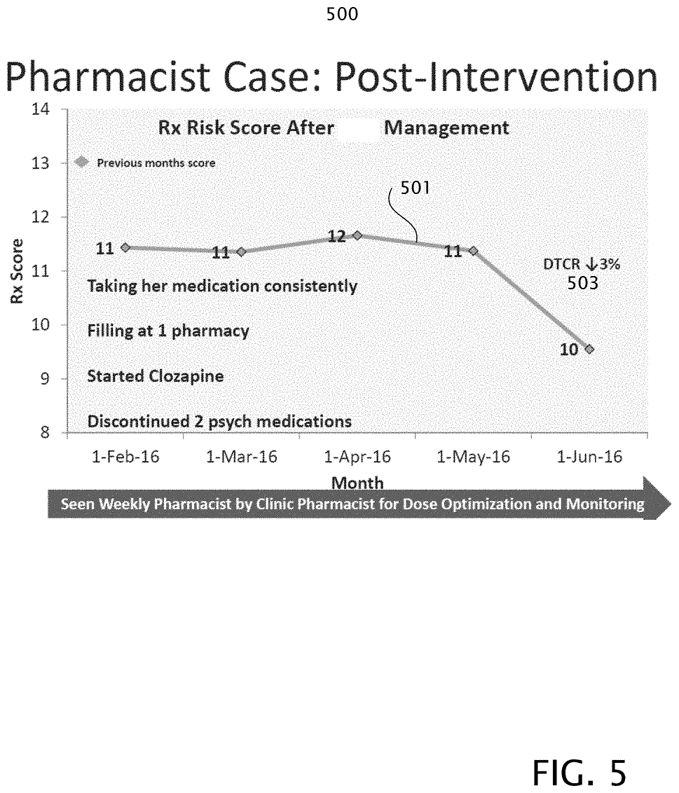

FIG. 5 is a graph plotting drug therapy coordination risk score against time for an exemplary patient of FIG. 4 after intervention 500. The scores in FIG. 4 caused an intervention program to be put into effect for the exemplary patient resulting in new data being obtained and plotted 501. Importantly, and advantageously for the patient the score has decreased 3%, 501 as progress was monitored and appropriate care put in place.

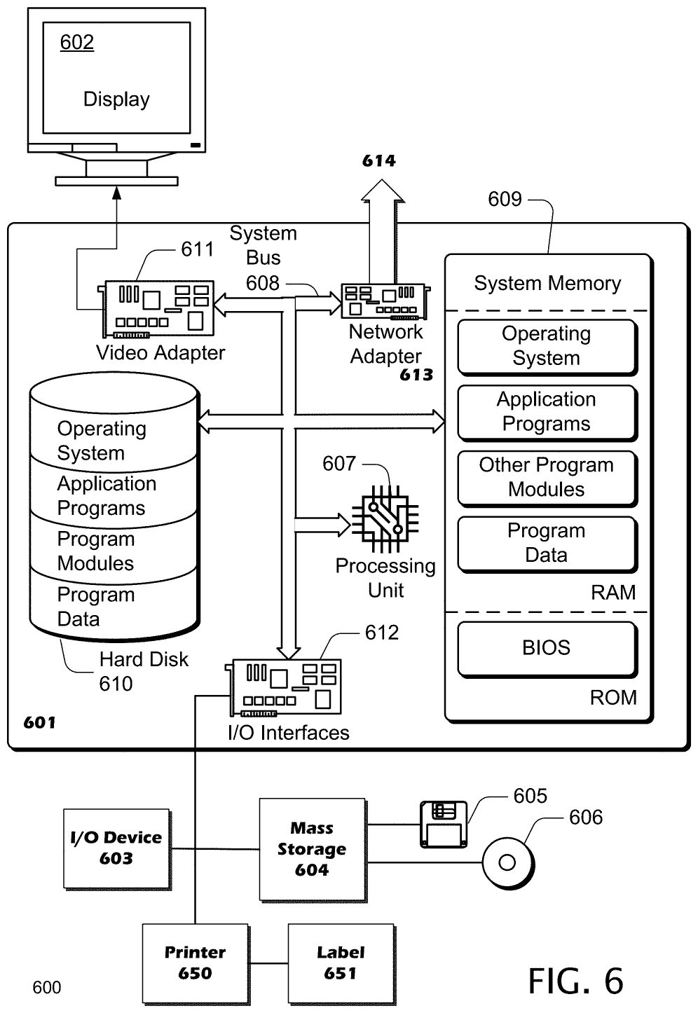

FIG. 6 illustrates an exemplary computing environment 600 in which the system and method for providing a drug therapy coordination risk score and an improved model of care (“pharmacy risk model”) described in this application, may be implemented. Exemplary computing environment 600 is only one example of a computing system and is not intended to limit the examples described in this application to this particular computing environment. For example the method for providing a drug therapy coordination risk score may be implemented in a PC, Laptop, tablet, a dedicated logic circuit, or “burned” into a programmable logic array using methods known to those skilled in the art.

For example the computing environment 600 can be implemented with numerous other general purpose or special purpose computing system configurations. Examples of well-known computing systems, may include, but are not limited to, personal computers, hand-held or laptop devices, microprocessor-based systems, multiprocessor systems, cellular telephones, PDAs, and the like.

The computer 600 includes a general-purpose computing system in the form of a computing device 601. The components of computing device 601 can include one or more processors (including CPUs, GPUs, microprocessors, dedicated logic circuits, programmable logic arrays (“PALs”) and the like 607, a system memory 609, and a system bus 608 that couples the various system components. Alternatively PALS and dedicated logic circuits (implementing a Boolean function to implement the process described herein) may be implemented as is known to those skilled in the art to in other input/output circuit configurations. Processor 607 processes various computer executable instructions, including those to implement a system and method for providing a drug therapy coordination risk score and an improved model of care (“pharmacy risk model”), to control the operation of computing device 601, and to communicate with other electronic and computing devices that may be present (not shown). The system bus 608 represents any number of several types of bus structures, including a memory bus or memory controller, a peripheral bus, an accelerated graphics port, and a processor or local bus using any of a variety of bus architectures.

The system memory 609 includes computer-readable media in the form of volatile memory, such as random access memory (RAM), and/or non-volatile memory, such as read only memory (ROM). A basic input/output system (BIOS) is stored in ROM. RAM typically contains data and/or program modules that are immediately accessible to and/or presently operated on by one or more of the processors 607.

Mass storage devices 604 may be coupled to the computing device 601 or incorporated into the computing device by coupling to the bus. Such mass storage devices 604 may include a magnetic disk drive which reads from and writes to a removable, non volatile magnetic disk (e.g., a “floppy disk”) 605, or an optical disk drive that reads from and/or writes to a removable, non-volatile optical disk such as a CD ROM or the like 606. Computer readable media 605, 606 typically embody computer readable instructions, data structures, program modules and the like supplied on floppy disks, CDs, portable memory sticks and the like.

Any number of program modules can be stored on the hard disk 610, Mass storage device 604, ROM and/or RAM 609, including by way of example, an operating system, one or more application programs, other program modules, and program data. Each of such operating system, application programs, other program modules and program data (or some combination thereof) may include an embodiment of the systems and methods described herein.

A display device 602 can be connected to the system bus 608 via an interface, such as a video adapter 611. A user can interface with computing device 702 via any number of different input devices 603 such as a keyboard, pointing device, joystick, game pad, serial port, and/or the like. These and other input devices are connected to the processors 607 via input/output interfaces 612 that are coupled to the system bus 608, but may be connected by other interface and bus structures, such as a parallel port, game port, and/or a universal serial bus (USB).

A printer 650 may be coupled to the computing device 601, typically via an IO Interface or the like 612 via an Ethernet or USB cable or equivalent. The printer 650 may be used to print out labels reports and the like 651 that may be used to convey the drug therapy coordination of risk score (“score”) by printing the score on reports, labels, or the like. The printer may be of any suitable type selected to provide a desired type of print out. However, in a first printer example a thermal printer, or equivalent, may be used to print out exemplary label 651 such as an adhesive backed label that include the drug therapy coordination of risk score and other indicia such as patient and medication information, bar coding and the like. For example a drug therapy coordination of risk score may be calculated when a prescription is refilled, with the score printed on the label that may be affixed to the medicine bottle. Alternatively when the score is calculated one or more adhesive backed labels may be printed with the score and affixed to charts reports and the like. In a further alternative printer example, the printer 650 may print on label stock such as labels with tamper resistant features, such as watermarks, or die cutting that destroys the label when one attempts to remove it, or the like.

Computing device 600 can operate in a networked environment using connections to one or more remote computers through one or more local area networks (LANs), wide area networks (WANs) and the like. Through such a networked structure, data for calculating a risk score may be obtained and the risk score transmitted to other users. The computing device 601 is connected to a network 614 via a network adapter 613 or alternatively by a modem, DSL, ISDN interface or the like.

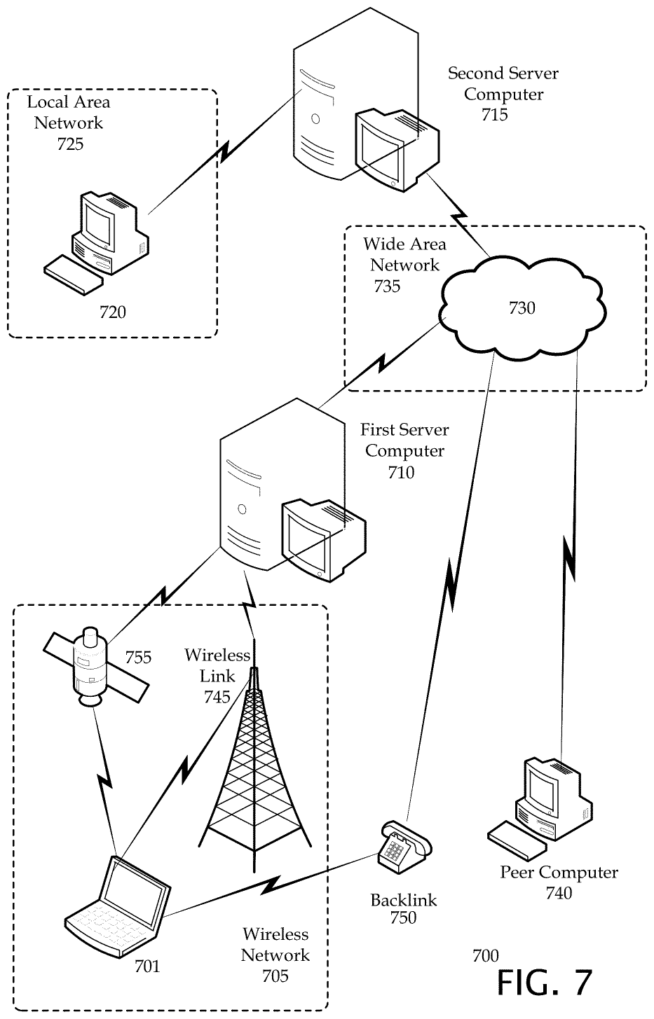

FIG. 7 is an exemplary network 700 in which the system and method for providing a drug therapy coordination risk score and an improved model of care (“pharmacy risk model”) may be implemented. Computer 715 may be a server computer coupled to a user's computer 720 through a conventionally constructed local area network 725.

In the local area network the user's computer is typically part of the local area network 725 which may include a plurality conventional computers (not shown) and conventional peripheral equipment (not shown) coupled together utilizing topologies (token, star and the like) and switching equipment known to those skilled in the art. Those skilled in the art will realize that other processor equipped devices such as cellular telephones, tablets, smart phones and the like may be coupled to the internet utilizing conventional techniques known to those skilled in the art.

A typical local area network 725 may include a conventionally constructed ISP network in which a number or plurality of subscribers utilize telephone dial up, ISDN, DSL, cellular telephone, cable modem, or the like connections to couple their computer to one or more server computers 715 that provide a connection to the world wide web 735 via the internet 730.

Wide area network or World Wide Web 735 is conventionally constructed and may include the internet 730 or equivalent coupling methods for providing a wide area network. As shown a conventionally constructed first server computer 710 is coupled to conventionally constructed second server computer 715 through a conventionally constructed internet connection to the World Wide Web 730.

In a peer to peer network a Peer computer 740 is conventionally constructed to couple to the internet 730 utilizing peer to peer network technology. Peer computer 740 may couple to a plurality of similarly connected peer computers in a peer to peer network (not shown), or to other computers 701, 720 that are part of conventionally constructed networks 725, 735.

In a conventional wireless network 705 a conventionally constructed computer 701 is coupled to the internet 730 via a conventionally constructed wireless link 745. The wireless link may include cellular, and satellite technology 755 to provide the link. Such a wireless network may include a conventionally constructed first server computer 710, typically provided to manage connections to a wide area network such as the internet. Those skilled in the art will realize that the computer 701 may be embodied as a processor coupled to the electronics of an automobile, and referred to as an automotive processor. Such a processor coupled to the internet may be used to find directions, provide medical data in case of an accident, first responder access of medical data (such as in an ambulance) report trouble or communicate with global positioning systems to determine position.

A conventionally constructed back link may be provided to efficiently provide an additional channel to couple to the internet. For example in situations where communication is one way in nature, the back link may provide communications in the opposite direction. An example would be viewing a listing of available on demand movies and ordering a selection via telephone 740. Those skilled in the art will realize that back links may equivalently be provided by cellular telephones, cordless telephones, paging devices and the like.

Those skilled in the art will realize that the process sequences described above may be equivalently performed in any order to achieve a desired result. Also, sub-processes may typically be omitted as desired without taking away from the overall functionality of the processes described above.

Those skilled in the art will realize that storage devices utilized to store program instructions can be distributed across a network. For example a remote computer may store an example of the process described as software. A local or terminal computer may access the remote computer and download a part or all of the software to run the program. Alternatively the local computer may download pieces of the software as needed, or distributively process by executing some software instructions at the local terminal and some at the remote computer (or computer network). Those skilled in the art will also realize that by utilizing conventional techniques known to those skilled in the art that all, or a portion of the software instructions may be carried out by a dedicated circuit, such as a DSP, programmable logic array, or the like.

1. A method to improve patient medication experience and health outcomes comprising:
obtaining pharmacy claim data for a patient filling a prescription;
evaluating a fundamental medication coordination status by calculating a drug therapy coordination of risk score from the pharmacy claim data that is proportional to a desired level of patient medication management;
printing a label and attaching it to a filled prescription including the drug therapy coordination of risk score;
transmitting the drug therapy coordination of risk score to a database for storage;
allocating use of emergency department and hospital facilities based on drug therapy coordination of risk scores from a plurality of patients obtained from the database; and
improving medication coordination risk based on drug therapy coordination of risk scores from a plurality of patients allowing prescribers, pharmacists and other healthcare support individuals to organize, screen and manage patients care.

2. A method to improve patient medication experience and health outcomes comprising:
calculating a drug therapy coordination of risk score from pharmacy claim data for a patient filling a prescription;
printing a thermal adhesive backed label for the prescription including the drug therapy coordination of risk score;
evaluating fundamental medication coordination based on the drug therapy coordination of risk score;
predicting future use of emergency department and hospital use based on the drug therapy coordination of risk score; and
improving medication coordination risk allowing prescribers, pharmacists and other healthcare support individuals to organize, screen and manage patients care.

3. The circuit implementing a method to improve patient medication experience and health outcomes of claim 2, in which the adhesive backed label is tamper resistant.

4. The circuit implementing a method to improve patient medication experience and health outcomes of claim 2, in which the adhesive backed label includes a bar code.

5. A method of reducing patient treatment risk based on historical pharmacy data comprising:
obtaining pharmacy claim data for a patient from a pharmacy data base records;
loading the pharmacy claim data for the patient into a processor;
calculating a drug therapy coordination of risk score, that is designed to be directly proportional an increased level of managed care, from a plurality data inputs taken from the pharmacy claim data for the patient;
thermally printing a tamper resistant label including the rug therapy coordination of risk score the patient
comparing the drug therapy coordination of risk score to a preset threshold value so that above the threshold a healthcare provider is alerted and a program of intensive medication management is established for the patient due to a scoring that is directly proportional to risk due to the level of medication the patient is receiving.

6. The method of reducing patient treatment risk based on historical pharmacy data of claim 5, in which the drug therapy coordination risk score (DTCR) is calculated from
DTCR=4.5−0.04(Distinct Fill Date)+0.34(Distinct GPI14Count)−0.31(Distinct GPI2Count)+0.31(Distinct Pharmacy Count)+0.56(Distinct Prescriber Count)−0.06(Average Days Supply),
where “Distinct Fill Date” is defined as Date Prescription was Filled; “Distinct GPI14Count” is defined as Generic Product Identifier from Medi-Span; a hierarchical therapeutic classification structure; “Distinct GPI2Count” is defined as derived from GPI14. “Distinct Pharmacy Count” is defined as the pharmacy's count; “Distinct Prescriber Count” is defined as the prescriber's count; “Average Days' Supply” is defined as Days of Drug Supply (e.g., 30 days); and 4.5 is an adjustment factor.

7. The method of reducing patient treatment risk based on historical pharmacy data of claim 5, in which a patient with a calculated drug therapy coordination risk score is diverted to one of a plurality of medication management programs to lessen their risk of complications indicated by the calculated drug therapy coordination risk score.

8. The method of reducing patient treatment risk based on historical pharmacy data of claim 5, in which a plurality of drug therapy coordination risk scores are calculated for a population of patients.

9. The method of reducing patient treatment risk based on historical pharmacy data of claim 5, in which a drug therapy coordination risk score of 9 to 14.99 causes the patient to be placed on a medication management program.

10. The method of reducing patient treatment risk based on historical pharmacy data of claim 5, in which a drug therapy coordination risk score of greater than 15 causes the patient to be placed on an intensive medication management program.

11. The method of reducing patient treatment risk based on historical pharmacy data of claim 5, in which the plurality of drug therapy coordination risk scores are used to predict future use of an emergency department.

12. The method of reducing patient treatment risk based on historical pharmacy data of claim 5, in which the plurality of drug therapy coordination risk scores are used to predict future use of a hospital.

13. The method of reducing patient treatment risk based on historical pharmacy data of claim 5, in which the plurality of drug therapy coordination risk scores provide a unit of measure to allow prescribers, pharmacists and other healthcare support individuals to organize, screen and manage patients care based on risk determined from patient medication data.
